(19) United States
(12) Patent Application Publication
(10) Pub. No.: US 2024/0317800 A1
(43) Pub. Date: Sep. 26, 2024

(54) NOVEL COMPOUNDS AND ANTIBACTERIAL COMPOSITION COMPRISING THE SAME

(71) Applicant: KOREA INSTITUTE OF OCEAN SCIENCE & TECHNOLOGY, Yeongdo-gu (KR)

(72) Inventors: Hee Jae SHIN, Haeundae-gu (KR); Hwa Sun LEE, Uiwang-si (KR)

(21) Appl. No.: 18/448,611

(22) Filed: Jun. 7, 2024

(30) Foreign Application Priority Data
Aug. 12, 2022 (KR) 10-2022-0101541

(51) Int. Cl. C07K 5/02; A61P 31/04

(52) U.S. Cl. CPC C07K 5/0215; A61P 31/04

(57) ABSTRACT

The present disclosure relates to a compound represented by Chemical Formula 1 and an antibacterial pharmaceutical composition, an antibacterial food composition, an antibacterial cosmetic composition or an antibacterial feed composition comprising the compound as an active ingredient, and has excellent inhibitory activity against Mycoplasma, and thus may be usefully used to prevent or treat infectious diseases caused by Mycoplasma.

### CROSS-REFERENCE TO RELATED APPLICATIONS

This application is based on and claims priority from Korean Patent Application No. 10-2022-0101541, filed on Aug. 12, 2022, with the Korean Intellectual Property Office, the disclosure of which is incorporated herein in its entirety by reference.

### TECHNICAL FIELD

The present disclosure relates to novel compounds and an antibacterial composition comprising the same.

### BACKGROUND

Marine microorganisms have been recognized as an important resource for novel compounds or bioactive materials. Marine Bacillus species produce structurally diverse secondary metabolites (such as lipopeptides, polypeptides, macrolides, fatty acids, polyketides, carotenoids, and isocoumarins), and these materials have various physiological activities (antibacterial, anticancer, antialgal, etc.). In particular, surfactins, iturins, fengycins, etc., which are cyclic lipopeptides derived from Bacillus subtilis, have strong antibacterial activity, and thus, receive much attention due to their high potential for application in biotechnology and pharmaceutical fields.

In previous studies, it was confirmed that marine-derived Bacillus subtilis 109GGC020 produced interesting secondary metabolites, such as gageomacrolactins which are macrolactins with antibacterial activity; gageotetrins A to C, gageopeptides A to D, and gageostatins A to C which are linear lipopeptides; and gageopeptins A and B and bacilotetrins A and B which are cyclic lipopeptides.

Linear lipopeptides such as gageopeptides A to D and gageotetrin B showed an inhibitory effect on wheat blast fungus, Magnaporthe oryzae Triticum, and based on this result, the corresponding compounds showed potential as agricultural antibiotics.

Meanwhile, Mycoplasma is generally known as the smallest bacteria, may survive without oxygen, and exists in various forms due to the absence of cell walls. Mycoplasma infects not only animals and plants, but also insects and humans, and is also often found as a contaminant of cell culture in laboratories. Among Mycoplasma spp., M. hyorhinis is a bacterium symbiotic with the upper respiratory tract of pigs and is also a pathogenic bacterium found in piglets. In addition, M. hyorhinis has been reported to cause polyserositis, arthritis, conjunctivitis, otitis, and a contaminant of cell culture.

Although many studies have been attempted to prevent or treat infectious diseases caused by Mycoplasma, research on new therapeutic agents is required because no commercially available drug has yet been developed that has clearly proven its effectiveness so that it can be applied to a wide range of mycoplasma infectious diseases.

Under this background, the development of a therapeutic agent having safer and excellent antibacterial effects has been required, and accordingly, the present inventors have completed the present disclosure by confirming that the compounds isolated from the Bacillus subtilis 109GGC020 strain isolated from Gageo Reef had an antibacterial effect on Mycoplasma.

### SUMMARY

The present disclosure has been made in an effort to provide a novel compound, an optical isomer thereof, or a pharmaceutically acceptable salt thereof.

The present disclosure has also been made an effort to provide an antibacterial composition including the novel compound, the optical isomer thereof, or the pharmaceutically acceptable salt thereof.

### Compound of Chemical Formula 1

An exemplary embodiment of the present disclosure provides a compound represented by Chemical Formula 1 below, an optical isomer thereof or a pharmaceutically acceptable salt thereof:

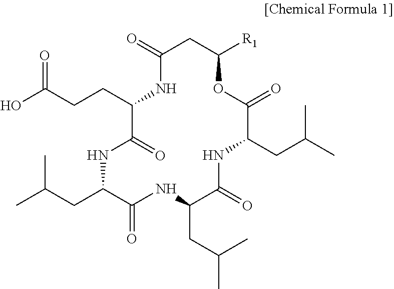

In Chemical Formula 1, R₁ is hydrogen, straight or branched C₁₋₂₀ alkyl, straight or branched C₁₋₂₀ alkenyl, straight or branched C₁₋₂₀ alkynyl, C₁₋₂₀ alkoxy, C₁₋₂₀ thioalkoxy, C₃₋₂₀ cycloalkyl, C₃₋₂₀ heterocycloalkyl, C₃₋₂₀ heteroaryl, phenyl, or halogen. Specifically, R₁ may be straight or branched C₁₋₁₅ alkyl. In this case, there is an excellent effect of inhibitory activity against Mycoplasma.

The compound of Chemical Formula 1 of the present disclosure may contain one or more asymmetric carbons and thus may exist as a racemate, a racemic mixture, a single enantiomer, a diastereomeric mixture, and an individual diastereomer.

These isomers are able to be separated by conventional techniques, and for example, the compound represented by Chemical Formula 1 is able to be separated by column chromatography or HPLC. Alternatively, each stereoisomer of the compound represented by Chemical Formula 1 may be stereospecifically synthesized using optically pure starting materials and/or reagents of known configuration.

According to an example embodiment of the present disclosure, the compound of Chemical Formula 1 may be represented by Chemical Formula 2 below:

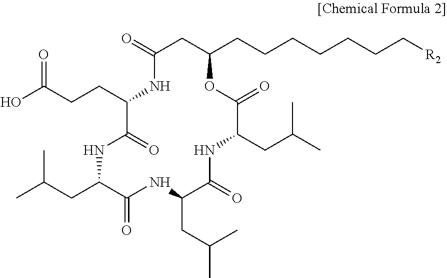

In Chemical Formula 2, R₂ is hydrogen, straight or branched C₁₋₁₃ alkyl, straight or branched C₁₋₁₃ alkenyl, straight or branched C₁₋₁₃ alkynyl, C₁₋₁₃ alkoxy, C₁₋₁₃ thioalkoxy, C₃₋₁₃ cycloalkyl, C₃₋₁₃ heterocycloalkyl, C₃₋₁₃ heteroaryl, phenyl, or halogen. Specifically, R₂ may be straight or branched C₁₋₅ alkyl. In this case, there is an excellent effect of inhibitory activity against Mycoplasma hyorhinis.

According to an exemplary embodiment of the present disclosure, the compound of Chemical Formula 1 may be cyclic lipodepsipeptide including three leucines, one glutamic acid, and p-hydroxy fatty acid.

According to an exemplary embodiment of the present disclosure, the compound of Chemical Formula 1 may be isolated from a microorganism deposited under the accession number KCTC 12411BP. The microorganism deposited under the accession number KCTC 12411 BP may be Bacillus subtilis 109GGC020 isolated from sponges collected in Gageo Reef, Korea.

According to an exemplary embodiment of the present disclosure, the compound of Chemical Formula 1 may be selected from the group consisting of compounds 1 to 3, as shown in Table 1 below. In this case, there is a significantly excellent effect of inhibitory activity against Mycoplasma hyorhinis.

TABLE 1

| Compound | Structure |
|---|---|
| 1 | 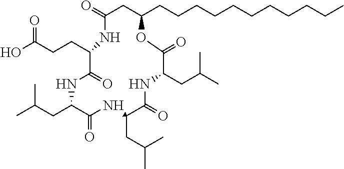 [SMILES: CCCCCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |
| 2 | 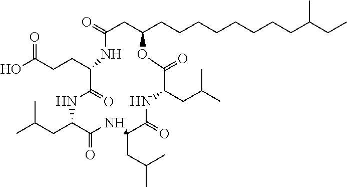 [SMILES: CCC(C)CCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |
| 3 | 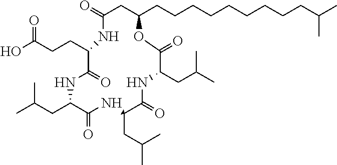 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |

In the present disclosure, the “pharmaceutically acceptable salt” refers to salts commonly used in the pharmaceutical industry. For example, the pharmaceutically acceptable salt includes inorganic ion salts prepared from calcium, potassium, sodium, magnesium, etc.; inorganic acid salts prepared from hydrochloric acid, nitric acid, phosphoric acid, hydrobromic acid, iodic acid, perchloric acid, sulfuric acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; sulfonic acid salts prepared from methanesulfonic acid, ethanesulfonic acid, benzenesulfonic acid, p-toluenesulfonic acid, naphthalenesulfonic acid, etc.; amino acid salts prepared from glycine, arginine, lysine, etc.; and amine salts prepared from trimethylamine, triethylamine, ammonia, pyridine, picoline, etc. However, the types of salts meant in the present disclosure are not limited to these listed salts.

### Preparation Method of Compound of Chemical Formula 1

Another exemplary embodiment of the present disclosure provides a method for preparing the compound of Chemical Formula 1, an optical isomer thereof, or a pharmaceutically acceptable salt thereof.

The preparation method may include isolating a compound of Chemical Formula 1 below, an optical isomer thereof, or a pharmaceutically acceptable salt thereof from a Bacillus subtilis 109GGC020 KCTC 12411BP strain or a culture thereof.

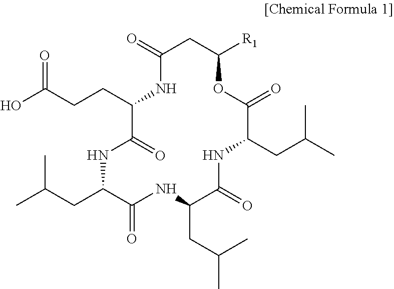

In Chemical Formula 1,

R₁ is hydrogen, straight or branched C₁₋₂₀ alkyl, straight or branched C₁₋₂₀ alkenyl, straight or branched C₁₋₂₀ alkynyl, C₁₋₂₀ alkoxy, C₁₋₂₀ thioalkoxy, C₃₋₂₀ cycloalkyl, C₃₋₂₀ heterocycloalkyl, C₃₋₂₀ heteroaryl, phenyl, or halogen.

In the present disclosure, the culture of the Bacillus subtilis 109GGC020 KCTC 12411BP strain may be obtained by culturing the strain in a liquid medium or solid medium. The medium may include, as a non-limiting example, glucose, starch syrup, dextrin, starch, molasses, animal oil, or vegetable oil as a carbon source. In addition, the medium may include, as a non-limiting example, bran, soybean meal, wheat, malt, cottonseed meal, fish scrap, cornstarch, broth, yeast extract, ammonium sulfate, sodium nitrate, or urea as a nitrogen source. In addition, the medium may contain salt, potassium, magnesium, cobalt, chlorine, phosphoric acid, sulfuric acid, or other inorganic salts that promote ion production, if necessary. The culture may be cultured while shaking or standing, and the culture temperature may be about 20° C. to about 37° C., preferably about 25° C. to about 30° C.

The compound of Chemical Formula 1 may be obtained by subjecting the strain or its culture to solvent extraction, concentration, and column chromatography. The concentration may be performed by adding a solvent to the strain or its culture and evaporating an extract under reduced pressure. As the solvent, ethyl acetate, chloroform, methylene chloride, and lower alcohols having 1 to 4 carbon atoms may be used, and preferably, ethyl acetate, chloroform, methylene chloride, and methanol may be used. The chromatography may be column chromatography, plate chromatography, paper chromatography, or thin-layer chromatography, depending on the form of a stationary phase. Alternatively, the chromatography may be high-performance liquid chromatography (HPLC) or gas chromatography, depending on the physical characteristics of a mobile phase.

According to an exemplary embodiment of the present disclosure, the compound of Chemical Formula 1 may be obtained by fractionation by vacuum column chromatography using an ethyl acetate extract of the strain or its culture and then purification by HPLC or reversed phase HLPC using a mixed solvent (i.e., aqueous methanol solution) of methanol and water.

Specifically, another exemplary embodiment of the present disclosure provides a method for preparing a compound, an optical isomer thereof, or a pharmaceutically acceptable salt thereof, including (a) culturing a Bacillus subtilis 109GGC020 KCTC 12411BP strain;
(b) extracting the culture medium of step (a) with ethyl acetate;
(c) stepwise-fractionating the extract of step (b) with a methanol aqueous solution (MeOH/H₂O), where the stepwise-fractionating is performed by sequential fractionation with a solvent having a v/v ratio of methanol (MeOH) to water (H₂O) of 1:4, 2:3, 3:2, 4:1, and 4:0, that is, a methanol aqueous solution of 20%, 40%, 60%, 80% and 100%;
(d) stepwise-fractionating the fractions in step (c) with MeOH/H₂O, where the stepwise elution is performed by fractionation with a solvent having a v/v ratio of methanol (MeOH) to water (H₂O) of 8:2, 9:1, and 10:0, that is, a methanol aqueous solution of 80%, 90%, and 100%; and
(e) purifying the compound of Chemical Formula 1 from the 90% methanol fraction or the 100% methanol fraction among the fractions in step (d).

### Use of Compound of Chemical Formula 1

Yet another exemplary embodiment of the present disclosure provides an antibacterial composition, including the compound of Chemical Formula 1, an optical isomer thereof, or a pharmaceutically acceptable salt thereof as an active ingredient.

According to an exemplary embodiment of the present disclosure, the compound of Chemical Formula 1 may have antibacterial activity against Mycoplasma.

Mycoplasma is a bacterium in an intermediate position between bacteria and viruses, belongs to the class Mollicutes without cell walls, and is a bacterium capable of self-reproduction in an artificial medium. For example, Mycoplasma is a bacterium widely distributed in humans, animals, insects, plants, etc., and may cause various diseases therefrom. Specifically, recently, it has been reported that Mycoplasma causes the synthesis of an important growth factor called BMP2 in lung cells of the human body and may convert normal lung cells into cells that cause tumors. In addition, Mycoplasma causes pneumonia, polyserositis, arthritis, conjunctivitis, ear disease, sepsis, or porcine genital respiratory syndrome in pigs and also causes infections in cattle and sheep. In plants, it is known to cause soft rot on cabbage, radish, and strawberry, and witches broom on jujube trees, chestnut trees, and peach trees. About 20 species of Mycoplasma spp. and Acholeplasma spp. are major sources of contamination in cell culture, and it has been reported that cell lines ranging from 5 to 35% are mainly contaminated by 6 species of Mycoplasma. The source of contamination is caused by animal tissues used for primary culture, serum used for culture, experimenters, or the like, and the contamination may spread to other cell lines due to cross-contamination of cell lines in a laboratory. In addition, Mycoplasma has no cell wall unlike bacteria having cell walls, so its shape changes easily, and since the diameter is as small as 0.2 to 2 m, Mycoplasma may pass through 0.22 to 0.45 m of a membrane filter used for filtration of a cell culture medium, Mycoplasma may be contaminated through the cell culture medium.

Mycoplasma spp. may be at least one selected from the group consisting of Mycoplasma hyorhinis, Mycoplasma yeastii, Mycoplasma equirhinis, Mycoplasma orale, Mycoplasma mycoides, Mycoplasma falconis, Mycoplasma arginini, Mycoplasma agalactiae, Mycoplasma felifaucium, Mycoplasma salivarium, Mycoplasma synoviae, Mycoplasma felis, Mycoplasma fermentans, Mycoplasma alkalescens, Mycoplasma gallinaceum, Mycoplasma hominis, Mycoplasma adleri, Mycoplasma gateae, Mycoplasma arthritidis, Mycoplasma alvi, Mycoplasma gypis, Mycoplasma pneumoniae, Mycoplasma anseris, Mycoplasma indiense, Mycoplasma pirum, Mycoplasma auris, Mycoplasma lagogenitalium, Mycoplasma spermatophilum, Mycoplasma bovigenitalium, Mycoplasma leonicaptivi, Mycoplasma buccale, Mycoplasma leopharyngis, Mycoplasma genitalium, Mycoplasma californicum, Mycoplasma lipofaciens, Mycoplasma hyosynoviae, Mycoplasma canadense, Mycoplasma molare, Mycoplasma pulmonis, Mycoplasma canis, Mycoplasma neurolyticum, Mycoplasma hyopneumoniae, Mycoplasma bovirhinis, Mycoplasma putrefaciens, Mycoplasma cottewii, Mycoplasma buteonis, Mycoplasma simbae, Acholeplasma laidlawii, Mycoplasma caviae, Mycoplasma testudinis, Acholeplasma oculi, Mycoplasma collis, Mycoplasma timone, Acholeplasma granularum, Spiroplasma citri, Ureaplasma urealyticum, Spiroplasma insolitum, Spiroplasma kunkelii, Ureaplasma parvum, Spiroplasma melliferum, Spiroplasma phoeniceum, and Spiroplasma mirum. Specifically, the compound of Chemical Formula 1 may have antibacterial activity against Mycoplasma hyorhinis.

According to an exemplary embodiment of the present disclosure, the composition may be a pharmaceutical composition.

The pharmaceutical composition according to the present disclosure may further include at least one pharmaceutically acceptable carrier in addition to the compound represented by Chemical Formula 1, the optical isomer thereof, or the pharmaceutically acceptable salt thereof for administration. The pharmaceutically acceptable carrier may be used by mixing saline, sterile water, Ringer's solution, buffered saline, a dextrose solution, a maltodextrin solution, glycerol, ethanol, and at least one of these ingredients, and if necessary, other conventional additives such as antioxidants, buffers, and bacteriostats may be added. In addition, the pharmaceutical composition may be prepared in injectable formulations such as aqueous solutions, suspensions, emulsions, pills, capsules, granules, or tablets by further adding a diluent, a dispersant, a surfactant, a binder, and a lubricant. Accordingly, the pharmaceutical composition of the present disclosure may be patches, liquids, pills, capsules, granules, tablets, suppositories, or the like. These formulations may be prepared by conventional methods used for formulation in the art or by methods disclosed in Remington's Pharmaceutical Science (latest edition), Mack Publishing Company, and Easton PA, and may be prepared into various formulations according to each disease or an ingredient.

The pharmaceutical composition of the present disclosure may be administered orally or parenterally (e.g., intravenously, subcutaneously, intraperitoneally, or topically) according to a desired method, and the range of the dose may vary depending on the body weight, age, sex, and health condition of a patient, a diet, an administration time, an administration method, an excretion rate, the type and severity of a disease, and the like. The daily dose of the compound of Chemical Formula 1 of the present disclosure is about 0.01 to 1,000 mg/kg, preferably 0.1 to 100 mg/kg, and may be administered once or several times a day.

The pharmaceutical composition according to the present disclosure may further include at least one active ingredient having the same or similar efficacy in addition to the compound represented by Chemical Formula 1, the optical isomer thereof or the pharmaceutically acceptable salt thereof.

According to an exemplary embodiment of the present disclosure, the composition may be a food composition.

The food composition according to the present disclosure may be used as a health functional food. The “health functional food” refers to food produced and processed using raw materials or ingredients with functionality, which are useful for the human body according to the Health Functional Foods Act No. 6727, and “functionality” means intake for adjusting nutrients for the structures and functions of the human body or obtaining a useful effect on health applications such as physiological actions.

The food composition according to the present disclosure may include conventional food additives, and the suitability as the “food additives” is determined by the specifications and standards for the corresponding item in accordance with the general rules, general test methods, and the like of the Food Additive Codex approved by the Food and Drug Administration, unless otherwise specified.

The food composition according to the present disclosure may include the compound of Chemical Formula 1 in an amount of 0.01% to 95 wt %, preferably 1 to 80 wt % based on the total weight of the composition, for the purpose of preventing and/or improving infectious diseases caused by microorganisms to be antibacterial of the antibacterial composition. In addition, the food composition may be produced and processed in the form of tablets, capsules, powders, granules, liquids, pills, and beverages for the purpose of preventing and/or improving infectious diseases.

According to an exemplary embodiment of the present disclosure, the composition may be a cosmetic composition.

The cosmetic composition according to the present disclosure may include the compound of Chemical Formula 1 in an amount of 0.01 to 95 wt %, preferably 1 to 80 wt % based on the total weight of the composition, for the purpose of preventing and/or improving infectious diseases caused by microorganisms to be antibacterial of the antibacterial composition. In addition, the cosmetic composition may include, without limitation, other commonly accepted ingredients in addition to the active ingredient, and may include conventional adjuvants such as antioxidants, stabilizers, solubilizers, vitamins, pigments and flavors, and carriers. The cosmetic composition may be interpreted as including various materials for skin health without limitation.

According to an exemplary embodiment of the present disclosure, the composition may be a feed composition.

The feed composition according to the present disclosure may further include known feed supplements, food additives, or feed additives, for the purpose of preventing and/or improving infectious diseases caused by microorganisms to be antibacterial of the antibacterial composition, and may be prepared in the form of fermented feed, formulated feed, pellet form, silage, and the like.

The antibacterial composition of the present disclosure may be preferably a quasi-drug composition. That is, according to the present disclosure, there is provided a quasi-drug composition for preventing or improving infectious diseases caused by pathogenic microorganisms or resistant bacteria. The quasi-drug composition of the present disclosure may be used together with other quasi-drugs or quasi-drug ingredients, and may be appropriately used according to a conventional method. The mixed amount of the active ingredients may be suitably determined according to a purpose of use (prevention, health, or therapeutic treatment). The quasi-drug composition may be a disinfectant cleanser, shower foam, mouthwash, wet tissue, detergent soap, hand wash, humidifier filler, mask, ointment, or filter filler, but is not limited thereto.

Yet another exemplary embodiment of the present disclosure provides a method for preventing or treating infectious diseases caused by microorganisms to be antibacterial of the antibacterial composition, including administering the antibacterial composition in a therapeutically effective dose to a subject other than humans.

The infectious diseases caused by the microorganisms to be antibacterial of the antibacterial composition may be at least one selected from the group consisting of polyserositis, arthritis, conjunctivitis, otitis, septicaemia, pneumonia, peritonitis, pleuritis, pericarditis, and porcine reproductive and respiratory syndrome (PRRS).

According to an exemplary embodiment of the present disclosure, the subject may be pigs. As described above, Mycoplasma hyorhinis may be a pathogen causing pneumonia, polyserositis, arthritis, conjunctivitis, ear disease, sepsis, or porcine reproductive and respiratory syndrome in pigs. The antibacterial composition has antibacterial activity against Mycoplasma hyorhinis, and thus may be applied as a pharmaceutical composition for preventing or treating the diseases occurring in pigs.

The term “therapeutically effective dose” used herein refers to an amount of the compound represented by Chemical Formula 1, the optical isomer or the pharmaceutically acceptable salt thereof, which is effective for preventing or treating infectious diseases caused by microorganisms to be antibacterial of the antibacterial composition.

The method of prevention or treatment of the present disclosure includes not only treating the disease itself before the onset of symptoms, but also inhibiting or avoiding symptoms thereof, by administering the compound of Chemical Formula 1. In the management of a disease, a preventive or therapeutic dose of a specific active ingredient will vary depending on the nature and severity of the disease or condition, and a route by which the active ingredient is administered. The dose and the dose frequency will vary according to the age, body weight, and response of an individual patient. A suitable dosage regimen may be easily selected by those skilled in the art who certainly consider these factors. In addition, the method of preventing or treating the diseases of the present disclosure may further include administering a therapeutically effective dose of an additional active agent to help in treating the diseases together with the compound of Chemical Formula 1, and the additional active agent may exhibit a synergistic or auxiliary effect together with the compound of Chemical Formula 1.

In the present disclosure, the “prevention” means any action to suppress infectious diseases caused by the pathogenic microorganisms or resistant bacteria or delay the onset thereof by administration of the composition. The “treatment” refers to any action that improves or beneficially changes symptoms caused by infectious diseases caused by the pathogenic microorganisms or resistant bacteria by administration of the composition. As used herein, the term “subject” refers to all animals, including humans, who have or may develop infectious diseases caused by the pathogenic microorganisms or resistant bacteria. In addition, the diseases may be effectively prevented or treated by administering the antibacterial composition or pharmaceutical composition of the present disclosure to a subject.

Yet another exemplary embodiment of the present disclosure provides a method for sterilization or bacteriostatic of microorganisms to be antibacterial of the antibacterial composition, including treating the antibacterial composition in vitro. The microorganism means pathogenic microorganisms or resistant bacteria.

In the present disclosure, “sterilization” means an action to kill microorganisms such as pathogenic microorganisms or resistant bacteria, and “bacteriostatic” means an action to suppress the growth and proliferation of microorganisms such as pathogenic microorganisms or resistant bacteria.

According to the exemplary embodiments of the present disclosure, the compound represented by Chemical Formula 1, the optical isomer or the pharmaceutically acceptable salt thereof exhibits excellent inhibitory activity against Mycoplasma, thereby exhibiting excellent effects in the prevention or treatment of infectious diseases caused by Mycoplasma.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram illustrating a process of fractionating Compounds 1 to 3.

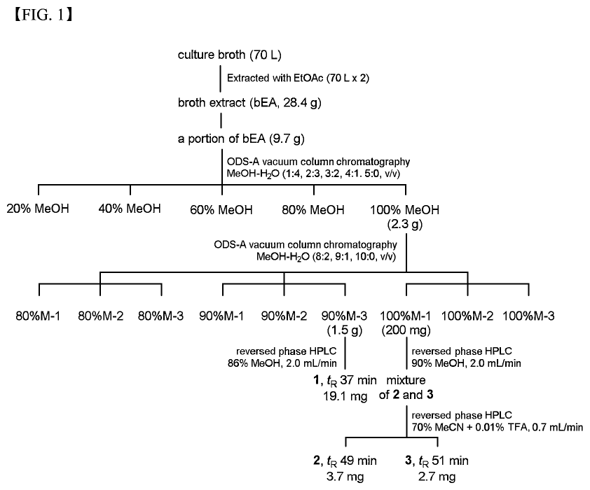

FIG. 2 illustrates chemical structures of Compounds 1 to 3 [1: Compound 1/2: Compound 2/3: Compound 3].

FIG. 3 illustrates partial structures and major 2D NMR correlations of Compounds 1 to 3.

FIG. 4 illustrates results of HPLC analysis of amino acids in Compound 1 through total hydrolysis.

FIG. 5 is a partial hydrolysis flowchart of Compound 1.

FIG. 6 illustrates results of HPLC analysis of L-FDLA derivatives of P1 and P3 hydrolysates.

FIG. 7 illustrates results of HPLC analysis of L-FDLA derivatives of P2.

FIG. 8 illustrates a value (ppm) of Δδ_{H}=δ_{S-ester}−δ_{R-ester} for MTPA ester of 1a.

FIG. 9 is an ¹H NMR spectrum (600 MHz, CD₃OH) of Compound 1.

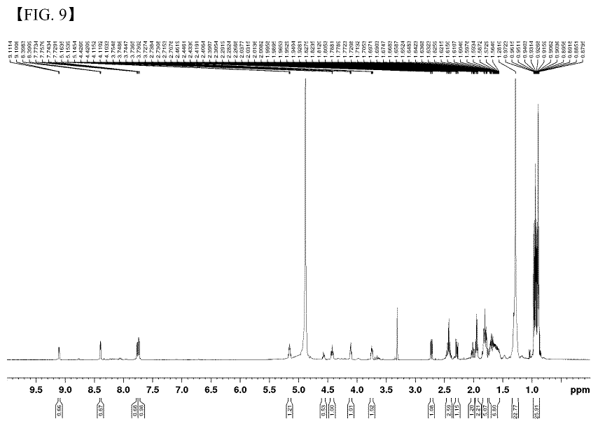

FIG. 10 is a ¹³C NMR spectrum (150 MHz, CD₃OH) of Compound 1.

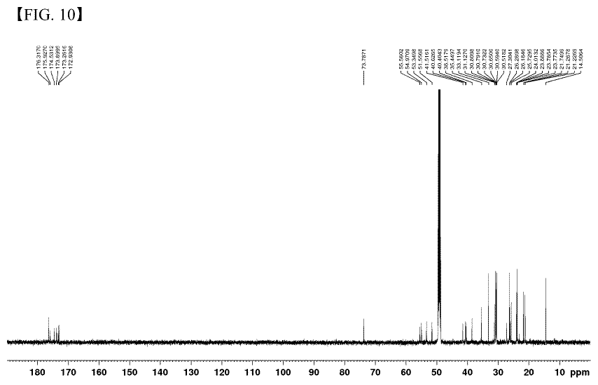

FIG. 11 is an HSQC spectrum of Compound 1 in CD₃OH.

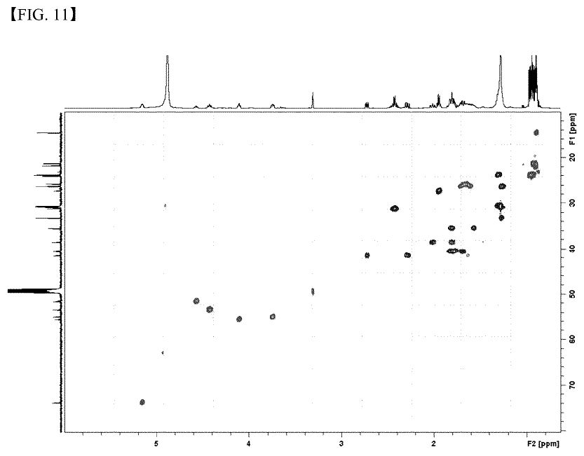

FIG. 12 is a COSY spectrum of Compound 1 in CD₃OH.

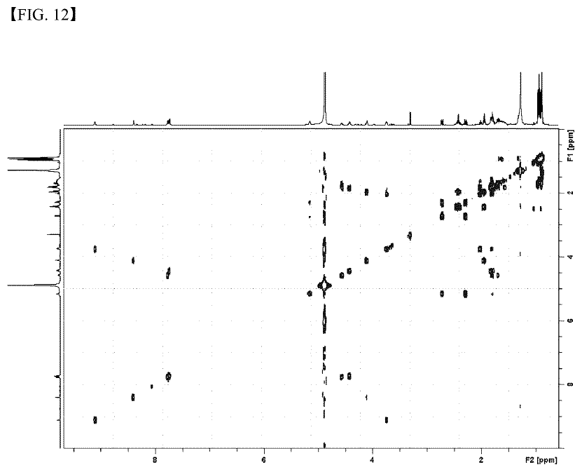

FIG. 13 is a TOCSY spectrum of Compound 1 in CD₃OH.

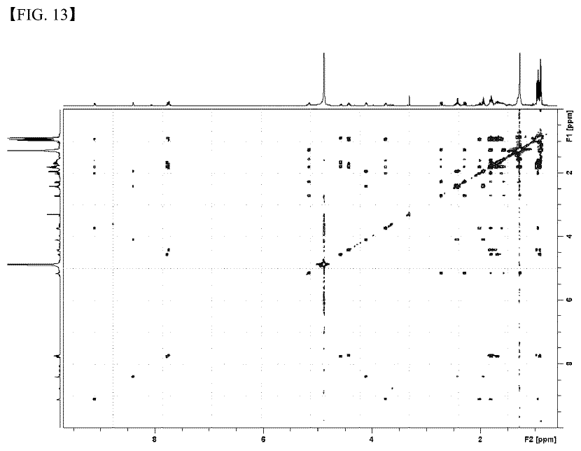

FIG. 14 is an HMBC spectrum of Compound 1 in CD₃OH.

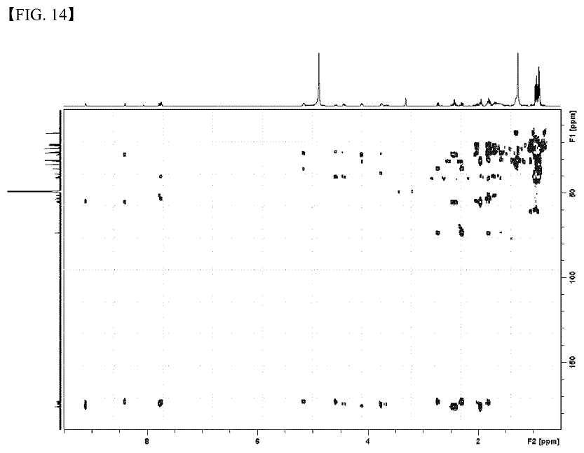

FIG. 15 is a NOESY spectrum of Compound 1 in CD₃OH.

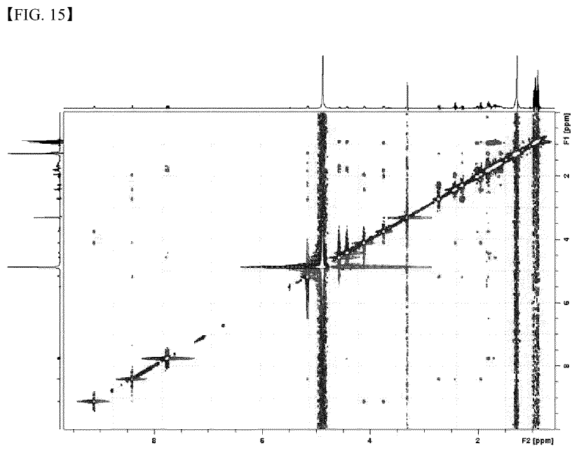

FIG. 16 is an HR-ESIMS spectrum of Compound 1.

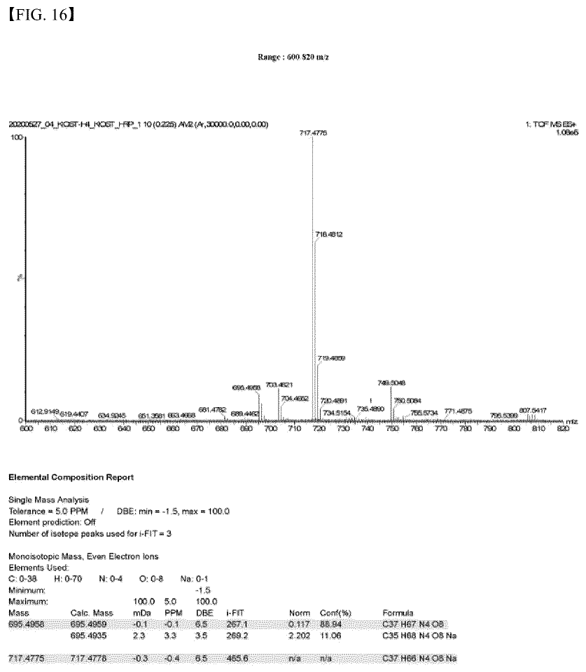

FIG. 17 is an ¹H NMR spectrum (600 MHz, CD₃OH) of Compound 2.

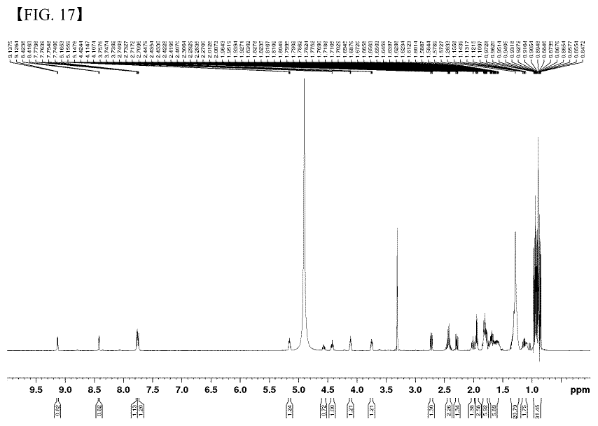

FIG. 18 is a ¹³C NMR spectrum (150 MHz, CD₃OH) of Compound 2.

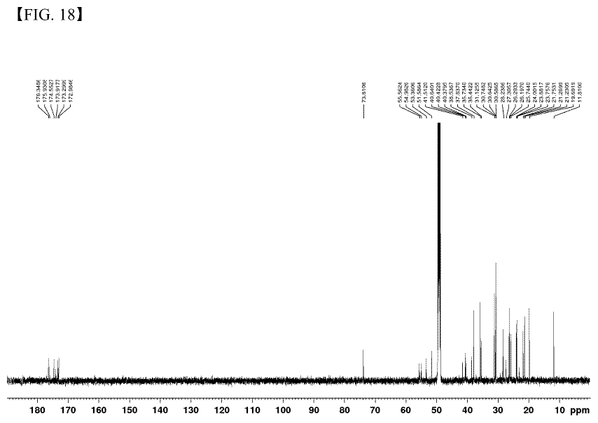

FIG. 19 is an HSQC spectrum of Compound 2 in CD₃OH.

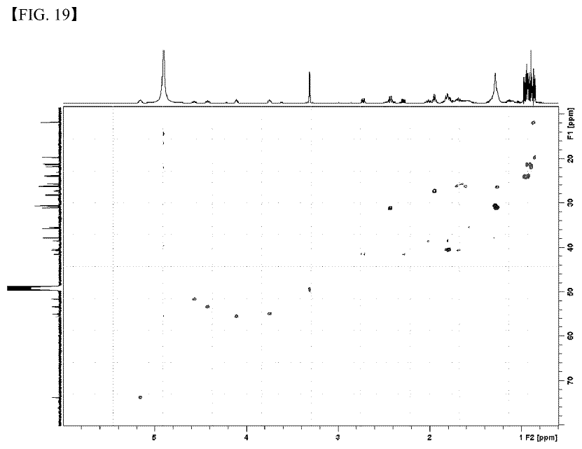

FIG. 20 is a COSY spectrum of Compound 2 in CD₃OH.

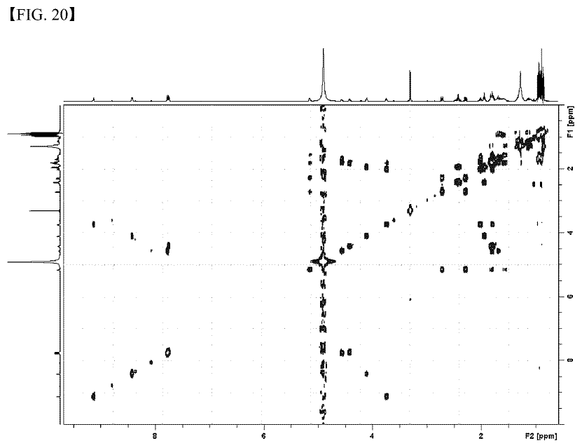

FIG. 21 is a TOCSY spectrum of Compound 2 in CD₃OH.

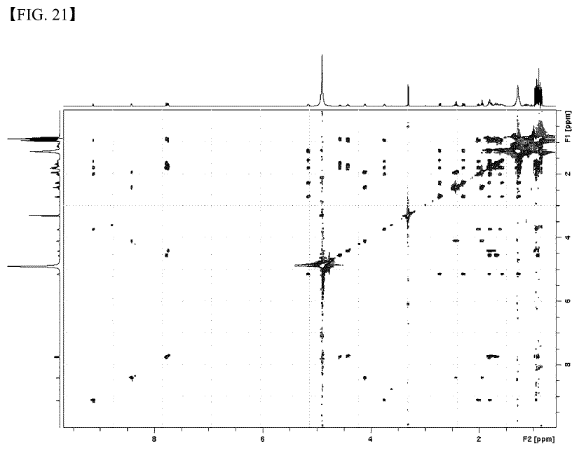

FIG. 22 is an HMBC spectrum of Compound 2 in CD₃OH.

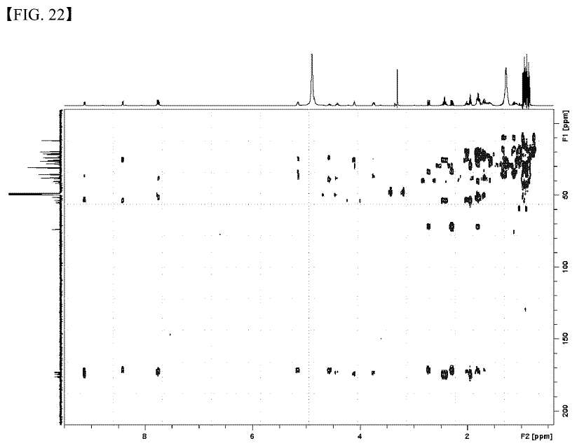

FIG. 23 is a ROESY spectrum of Compound 2 in CD₃OH.

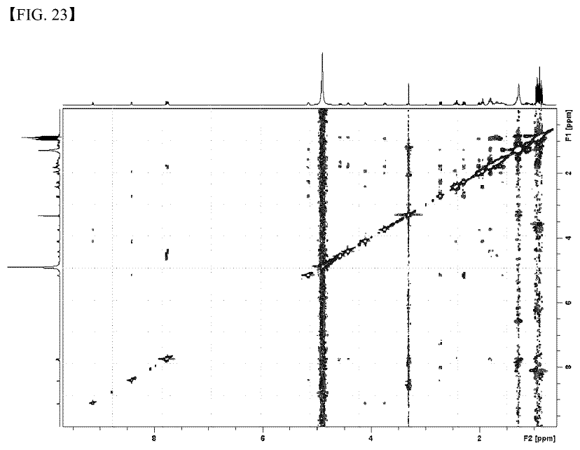

FIG. 24 is an HR-ESIMS spectrum of Compound 2.

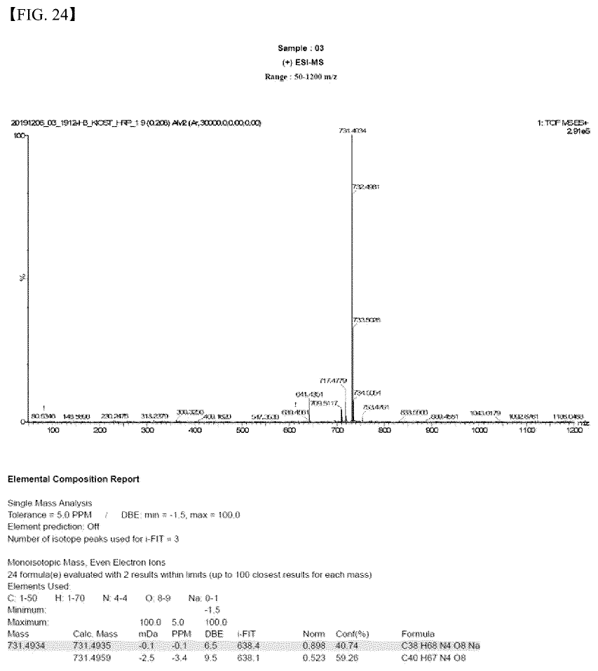

FIG. 25 is an ¹H NMR spectrum (600 MHz, CD₃OH) of Compound 3.

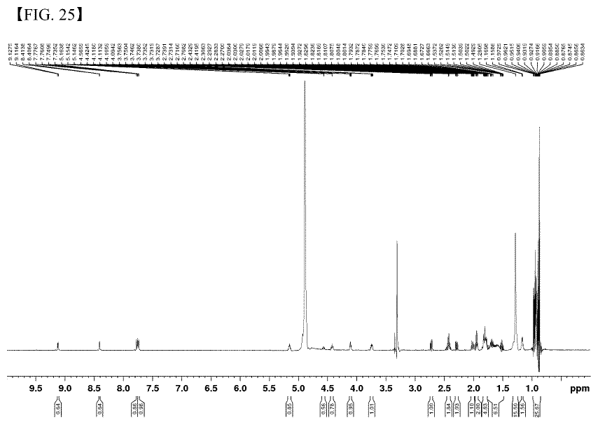

FIG. 26 is a ¹³C NMR spectrum (150 MHz, CD₃OH) of Compound 3.

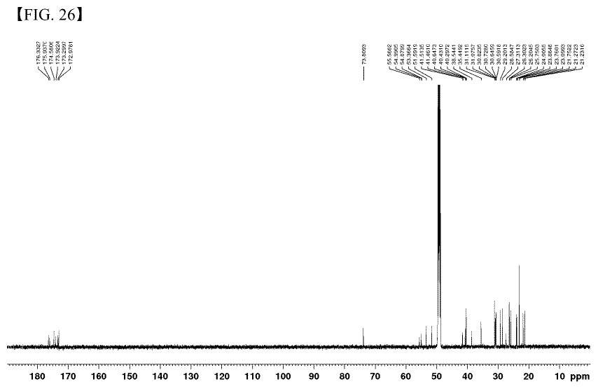

FIG. 27 is an HSQC spectrum of Compound 3 in CD₃OH.

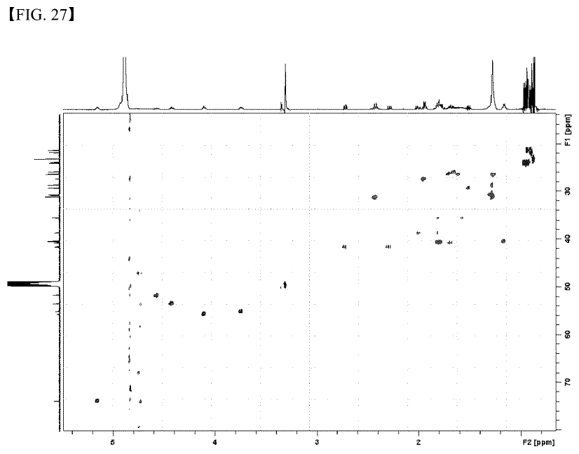

FIG. 28 is a COSY spectrum of Compound 3 in CD₃OH.

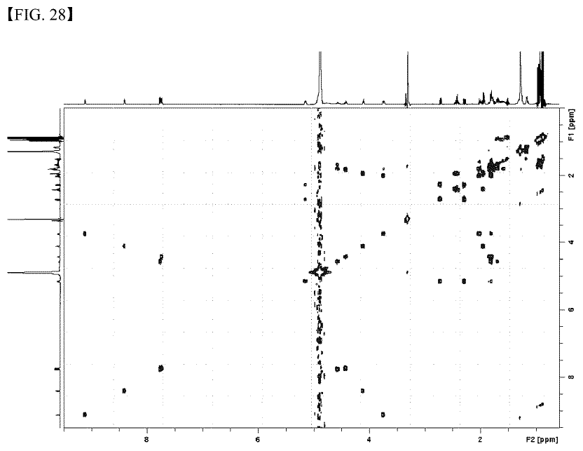

FIG. 29 is a TOCSY spectrum of Compound 3 in CD₃OH.

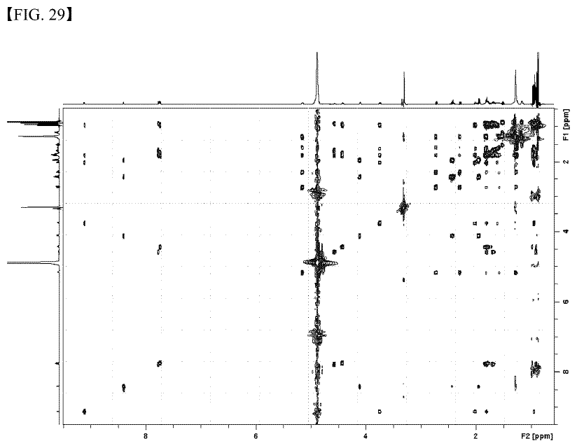

FIG. 30 is an HMBC spectrum of Compound 3 in CD₃OH.

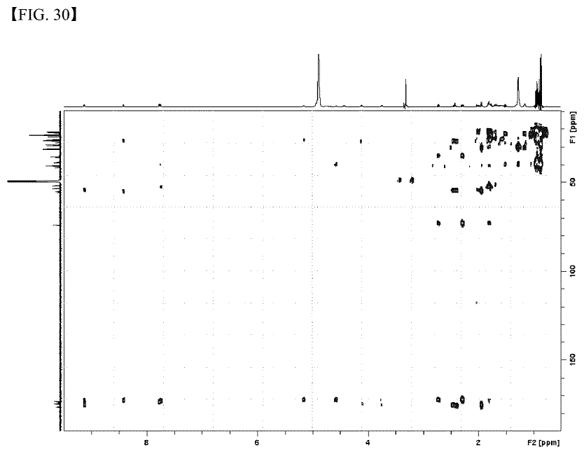

FIG. 31 is a ROESY spectrum of Compound 3 in CD₃OH.

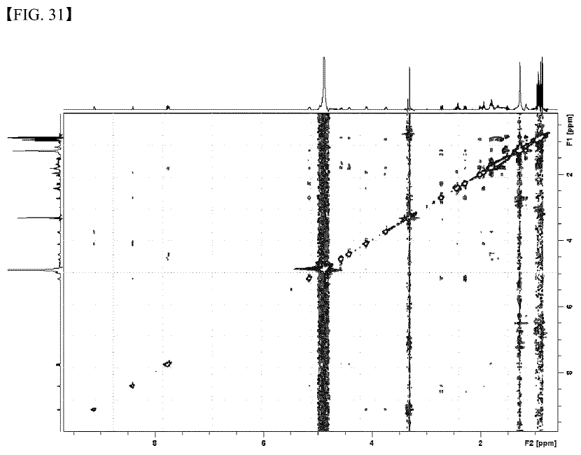

FIG. 32 is an HR-ESIMS spectrum of Compound 3.

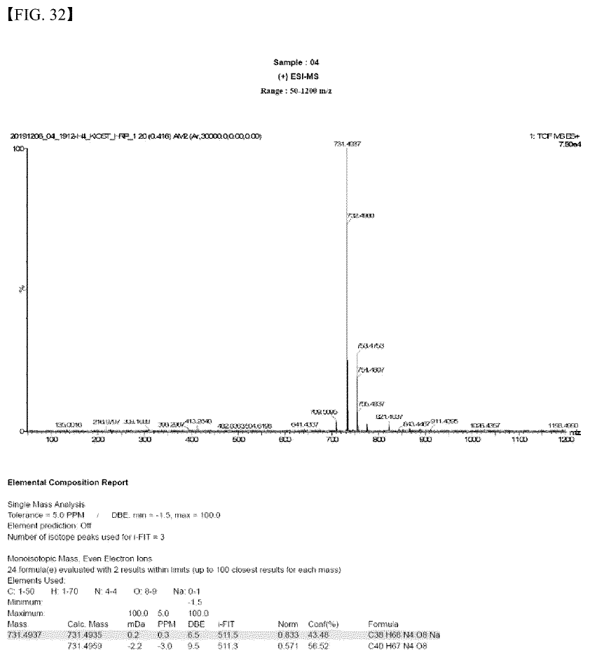

### DETAILED DESCRIPTION

Hereinafter, Examples of the present disclosure will be described in detail so as to easily implement those skilled in the art. However, the present disclosure may be embodied in many different forms and is limited to embodiments described herein.

In previous studies, cyclic lipopeptide-based bacilotetrins A and B having antibacterial activity were found from marine-derived Bacillus subtilis 109GGC020. Through additional research on an ethyl acetate (EtOAc) extract obtained from a culture of Bacillus subtilis 109GGC020, it was confirmed that three novel cyclic lipodepsipeptides, bacilotetrins C, D, and E (Compounds 1 to 3) were isolated, and these materials exhibited excellent antibacterial activity against Mycoplasma.

Hereinafter, the isolation, structural description, and antibacterial activity of bacilotetrins C, D, and E (Compounds 1 to 3), which were novel cyclic lipodepsipeptides, will be described.

### Experimental Materials and Apparatus

UV spectrum was obtained using a UV-1650PC spectrophotometer (Shimadzu Co., Japan). IR spectrum was measured using an FT/IR-4100 spectrophotometer (JASCO Co., Japan). Specific optical rotation was measured using an Autopol III S2 polarimeter (Rudolph analytical Co., USA). NMR spectra were measured at 600 MHz for ¹H and 150 MHz for ¹³C using a Bruker AVANCE III 600 spectrometer (Bruker BioSpin GmbH, Germany) and obtained using a 3 mm probe. Chemical shift values were based on solvent peaks (δ_{H} 3.31 and δ_{C} 49.15) of CD₃OH. LR-EIMS and Marfey's analysis were obtained using an Agilent 6100 single quadrupole mass spectrometer (Agilent Technologies, USA), and HR-ESIMS data were obtained using a SYNPT G2 Q-TOF mass spectrometer (Waters Co., USA) at the Korea Basic Science Institute (KBSI) in Cheongju, Korea. HPLC was used with a PrimeLine binary pump (Analytical Scientific Instruments, Inc., USA), a Shodex RI-101 refractive index detector (Shoko Scientific Co. Ltd., Japan), and an S3210 variable UV detector (Schambeck SFD GmbH, Germany), and Thermo Fisher Scientific UltiMate 3000 UHPLC (Thermo Scientific, Germany) was also used in the experiments. HPLC columns were used with YMC-ODS-A (250 mm×10 mm, 5 μm and 250 mm×4.6 mm, 5 μm), and YMC-Triart C₁₈ (250 mm×10 mm, 5 μm and 250 mm×4.6 mm, 5 μm). As a filler for open column chromatography, reversed-phase silica gel (YMC-Gel ODS-A, 12 nm, S-75 μm) was used. The organic solvents used in the experiment were HPLC-grade solvents purchased from Duksan (Korea) and Samchun (Korea). Distilled water and ultrapure water were obtained through a Milipore Mili-Q Direct 8 system (Milipore S.A.S., France).

### Examples

### Isolation and Culture of the Producing Strain

Bacillus subtilis 109GGC020 (KCTC 12411BP) was isolated from sponges collected from Gageo Reef, Korea in 2010.

For seed culture and mass cultures, a Bennett (BN) liquid medium (1% glucose, 0.2% tryptone, 0.1% yeast extract, 0.1% beef extract, 0.5% glycerol, 1.85% artificial sea salt, pH 7) was used. The seed culture was inoculated with cells in a 250 mL conical flask containing 100 mL of the BN liquid medium, and then cultured for 3 days in a shaking incubator at 28° C. and 120 rpm. For the mass culture, 70 L of the same liquid medium was prepared in a 100 L fermentor, a seed culture medium was inoculated in an aseptic condition, and then cultured at 28° C., 55 rpm, and airflow rate of 20 L/min (LPM) for 7 days. The culture medium was separated into cells and the culture medium using a high-speed centrifuge, and the separated culture medium was extracted twice with the same amount of ethyl acetate (EtOAc, 70 L×2).

Bacillus subtilis 109GGC020 was deposited on May 27, 2013 and was given accession number KCTC 12411BP.

### Separation and Purification of Compounds

The ethyl acetate (EtOAc) extract prepared from the strain culture medium was concentrated under reduced pressure to obtain 28.4 g of a crude extract. 9.7 g of the crude extract was subjected to vacuum column chromatography using ODS-A gel (YMC Gel ODS-A, 12 nm, S 75 μm). A solvent was eluted with a mixture of methanol and water stepwise (20, 40, 60, 80, and 100% MeOH in H₂O). 2.3 g of the 100% methanol fraction was subjected to ODS-A vacuum column chromatography once more, and sequentially eluted with the solvent conditions of 80, 90, and 100% MeOH. Each fraction was divided into three subfractions, and the third subfraction (1.5 g) of 90% MeOH was separated and purified using reversed-phase HPLC (YMC ODS-A, 250×10 mm, 5 μm, 86% MeOH, 2.0 mL/min, RI detector) to obtain Compound 1 (19.1 mg, t_{R} 37 min). The first subfraction (200 mg) of 100% MeOH fraction was purified using reversed-phase HPLC (YMC Triart C₁₈, 250×10 mm, 5 μm, 90% MeOH, 2.0 mL/min, RI detector) to obtain a small subfraction containing Compounds 2 and 3. This small subfraction was again subjected to reversed-phase HPLC (YMC Triart C₁₈, 250×4.6 mm, 5 m, 70% MeCN+0.010% TFA, 0.7 mL/min, UV detector: 224 nm) to separate and obtain pure compound 2 (3.7 mg, t_{R} 49 min) and compound 3 (2.7 mg, t_{R} 51 min).

Compound 1 (Bacilotetrin C (1)): amorphous solid; [α]_{D}²⁵−50 (c 0.1, MeOH); IR(MeOH) γₘₐₓ 3297, 2925, 1643, 1052 cm⁻¹; ¹H and ¹³C NMR data (Table 2); HR-ESIMS m/z [M+Na]⁺717.4775 (calculated for C₃₇H₆₆N₄O₈Na, 717.4778).

Compound 2 (Bacilotetrin D (2)): amorphous solid; [α]_{D}²⁵−70 (c 0.1, MeOH); IR(MeOH) γₘₐₓ 3300, 2957, 1653, 1057 cm⁻¹; ¹H and ¹³C NMR data (Table 2); HR-ESIMS m/z [M+Na]⁺731.4934 (calculated for C₃₈H₆₈N₄O₈Na, 731.4935).

Compound 3 (Bacilotetrin E (3)): amorphous solid; [α]_{D}²⁵−63 (c 0.1, MeOH); IR(MeOH) γₘₐₓ 3297, 2961, 1650, 1057 cm⁻¹; ¹H and ¹³C NMR data (Table 2); HR-ESIMS m/z [M+Na]⁺731.4937 (calculated for C₃₈H₆₈N₄O₈Na, 731.4935).

TABLE 2

| | 1 | | 2 | | 3 | |
|---|---|---|---|---|---|---|
| | δ_{C}, | δ_{H}, mult. | δ_{C}, | δ_{H}, mult. | δ_{C}, | δ_{H}, mult. |
| Position | type | (J in Hz) | type | (J in Hz) | type | (J in Hz) |

Glu

| | | | | | | |
|---|---|---|---|---|---|---|
| 1 | 175.9 | | 175.9, C | | 175.9, C | |
| 2 | 55.6, CH | 4.11, td (7.3, 4.0) | 55.5, CH | 4.11, m | 55.5, CH | 4.11, m |
| 3 | 27.3, CH₂ | 1.95, q (14.9, 7.3) | 27.3, CH₂ | 1.95, q (14.9, 7.4) | 27.3, CH₂ | 1.94, q (14.8, 7.8) |
| 4 | 31.1, CH₂ | 2.42, m | 31.1, CH₂ | 2.43, m | 31.1, CH₂ | 2.42, m |
| 5 | 176.3, C | | 176.3, C | | 176.3, C | |
| NH | | 8.39, d (4.7) | | 8.42, d (4.5) | | 8.41, d (4.5) |

Leu-1

| | | | | | | |
|---|---|---|---|---|---|---|
| 1 | 173.9, C | | 173.9, C | | 173.9, C | |
| 2 | 55.0, CH | 3.74, m | 55.0, CH | 3.75, m | 55.0, CH | 3.74, m |
| 3 | 38.5, CH₂ | 2.01, ddd (14.6, 11.0, 3.9) | 38.5, CH₂ | 2.01, m | 38.5, CH₂ | 2.01, m |
| | | 1.80, o.1^{a} | | 1.80, o.1^{a} | | 1.80, o.1^{a} |
| 4 | 26.3, CH | 1.61, m | 26.3, CH₂ | 1.60, o.1^{a} | 26.3, CH | 1.60, o.1^{a} |
| 5 | 21.3, CH₃ | 0.93, d (5.8) | 21.3, CH₃ | 0.93, d (5.4) | 21.3, CH₃ | 0.94, d (5.3) |
| 6 | 24.0, CH₃ | 0.95, d (5.8) | 24.0, CH₃ | 0.95, d (5.4) | 24.0, CH₃ | 0.95, d (5.3) |
| NH | | 9.10, d (6.7) | | 9.13, d (6.6) | | 9.12, d (6.7) |

Leu-2

| | | | | | | |
|---|---|---|---|---|---|---|
| 1 | 174.5, C | | 174.5, C | | 174.5, C | |
| 2 | 53.4, CH | 4.43, m | 53.3, CH | 4.42, m | 53.4, CH | 4.42, m |
| 3 | 40.4, CH₂ | 1.81, o.1^{a} | 40.4, CH₂ | 1.76, o.1^{a} | 40.4, CH₂ | 1.78, o.1^{a} |
| 4 | 26.2, CH | 1.70, m | 26.2, CH | 1.71, o.1^{a} | 26.2, CH | 1.71, o.1^{a} |
| 5 | 21.2, CH₃ | 0.90, d (6.4) | 21.2, CH₃ | 0.90, d (6.4) | 21.2, CH₃ | 0.91, d (6.4) |
| 6 | 23.9, CH₃ | 0.96, d (6.4) | 23.9, CH₃ | 0.96, d (6.4) | 23.9, CH₃ | 0.98, d (6.4) |
| NH | | 7.74, d (8.6) | | 7.75, d (8.5) | | 7.74, d (8.8) |

Leu-3

| | | | | | | |
|---|---|---|---|---|---|---|
| 1 | 172.9, C | | 173.0, C | | 173.0, C | |
| 2 | 51.6, CH | 4.57, m | 51.6, CH | 4.57, m | 51.6, CH | 4.57, m |
| 3 | 40.6, CH₂ | 1.80, m | 40.6, CH₂ | 1.81, m | 40.6, CH₂ | 1.80, m |
| | | 1.69, m | | 1.69, m | | 1.70, m |
| 4 | 25.8, CH | 1.65, o.1^{a} | 25.7, CH | 1.65, o.1^{a} | 25.7, CH | 1.66, m |
| 5 | 21.8, CH₃ | 0.89, o.1^{a} | 21.7, CH₃ | 0.89, d (6.4) | 21.7, CH₃ | 0.90, d (6.5) |
| 6 | 23.8, CH₃ | 0.92, d (6.5) | 23.7, CH₃ | 0.92, d (6.4) | 23.7, CH₃ | 0.93, d (6.5) |
| NH | | 7.76, d (9.5) | | 7.77, d (9.5) | | 7.77, d (9.4) |

β-OH acid

| | | | | | | |
|---|---|---|---|---|---|---|
| 1 | 173.3, C | | 173.3, C | | 173.0, C | |
| 2 | 41.5, CH₂ | 2.72, dd (13.8, 4.6) | 41.5, CH₂ | 2.72, dd (13.8, 4.6) | 41.5, CH₂ | 2.72, dd (13.8, 4.7) |
| | | 2.29, dd (13.8, 8.1) | | 2.29, dd (13.8, 8.1) | | 2.29, dd (13.8, 8.1) |
| 3 | 73.8, CH | 5.16, tt (7.8, 5.3) | 73.8, CH | 5.15, m | 73.8, CH | 5.15, m |
| 4 | 35.5, CH₂ | 1.80, o.1^{a} | 35.4, CH₂ | 1.81, o.1^{a} | 35.4, CH₂ | 1.81, o.1^{a} |
| | | 1.57, o.1^{a} | | 1.56, o.1^{a} | | 1.62, o.1^{a} |
| 5 | | | | | 26.3, CH₂ | 1.28, o.1^{a} |
| 6 | | | | | | |
| 7 | | | 28.2- | 1.29, o.1^{a} | | |
| 8 | 26.4- | | 30.6, CH₂ | | | |
| 9 | 30.8, CH₂ | 1.29, o.1^{a} | | | 28.6- | 1.28, o.1^{a} |
| 10 | | | | | 31.0, CH₂ | |
| 11 | | | 30.7, CH₂ | 1.34, o.1^{a} | | |
| | | | | 1.13, o.1^{a} | | |
| 12 | 33.1, CH₂ | 1.27, o.1^{a} | 35.7, CH | 1.29, o.1^{a} | 40.3, CH₂ | 1.16, m |
| 13 | 23.8, CH₂ | 1.30, o.1^{a} | 37.8, CH₂ | 1.29, o.1^{a} | 29.2, CH | 1.51, m |
| | | | | 1.09, o.1^{a} | | |
| 14 | 14.5, CH₃ | 0.89, o.1^{a} | 11.8, CH₃ | 0.87, o.1^{a} | 23.1, CH₃ | 0.87, d (6.4) |
| 15 | | | 19.7, CH₃ | 0.85, d (4.8) | 23.1, CH₃ | 0.87, d (6.4) |

^{a}Signals were overlapped with other signals

### Total Hydrolysis and Marfey's Analysis

Compound 1 (0.4 mg) was added with 6N HCl (300 μL) and stirred at 110° C. for 12 hours. The completion of the reaction was confirmed by LR-LCMS analysis, and the reactant was cooled at room temperature and fractionated with water and hexane. A water layer was concentrated under reduced pressure, added with 600 μL of 0.1% 1-fluoro-2,4-dinitro-phenyl-5-L-leucinamide (L-FDLA) dissolved in acetone and 120 μL of 1 M NaHCO₃ and then stirred at 40° C. for 1 hour. The mixture was cooled to room temperature, added with 120 μL of 1N HCl to be neutralized, and then diluted with MeCN (420 μL). Standard L- and D-amino acids were reacted with L-FDLA in the same manner. A Marfey's derivative of compound 1 was analyzed using LR-LCMS (YMC ODS-A, 250×4.6 mm, 5 μm, 0.5 mL/min, UV: 340 nm) under a MeCN—H₂O (+0.02% TFA) solvent system with a gradient condition (40% MeCN 5 min, 40-80% MeCN 20 min, 80% MeCN 5 min), and then compared with retention times of standard amino acid derivatives. As a result, the composition of amino acids included in compound 1 was identified as L-Glu (16.9 min), L-Leu (23.6 min), and D-Leu (29.0 min). The retention times of standard amino acid derivatives bound with L-FDLA were L-Glu (16.9 min), D-Glu (17.8 min), L-Leu (23.6 min), and D-Leu (28.9 min).

### Partial Hydrolysis and Marfey's Analysis

Compound 1 (2.0 mg) was added with 1 mL of 4N HCl:AcOH (1:1) and then reacted at 100° C. for 2 hours. The reactant was monitored using LR-LCMS, and the reactant for which partial hydrolysis was confirmed was concentrated with nitrogen gas and then fractionated using water and hexane. A concentrated water layer was eluted using LR-LCMS (YMC-ODS-A, 250×4.6 mm, 5 m, 0.5 mL/min, UV: 224 nm) under a MeCN—H₂O (+0.02% TFA) solvent condition with a gradient (20% MeCN 10 min, 20-100% MeCN 40 min, 100% MeCN 10 min), and separated into three partial structures P1: Glu-Leu. t_{R} 7.6 min, m/z 261 [M+H]⁺; P2: Leu-Leu, t_{R} 23.0 min, m/z 245 [M+H]⁺; P3: β-OH acid-Leu, t_{R} 28.6 min, m/z 358 [M+H]⁺). Among them, P1 (Glu-Leu) and P3 (β-OH acid-Leu) were subjected to total hydrolysis once more, reacted with L-FDLA, and analyzed using LR-LCMS as described above, and as a result, leucine contained in these two substructures was confirmed as L-form (hydrolyzate of P1-L-FDLA: t_{R} 23.7 min, hydrolyzate of P3-L-FDLA: t_{R} 23.7 min). The remaining partial structure P2 (Leu-Leu) reacted with L-FDLA and then compared and analyzed for retention times with the standard reagents (L-Leu-D-Leu and D-Leu-L-Leu) reacted with L-FDLA. It was confirmed that Leu-Leu of P2 was a mixture of L-Leu-D-Leu and D-Leu-L-Leu (P2-L-FDLA: t_{R} 26.1 min (major) and t_{R} 31.6 min (minor), m/z 589 [M+H]⁺; L-Leu-D-Leu-L-FDLA: t_{R} 26.1 min, m/z 589 [M+H]⁺, D-Leu-L-Leu-L-FDLA: t_{R} 31.6 min, m/z 589 [M+H]⁺).

### Methanolysis of Compound 1

Compound 1 (2.4 mg) was dissolved in 1.2 mL of 3 M methanolic HCl, and then refluxed for 2 hours. The completion of the reaction was confirmed through LR-LCMS analysis, and the reactant was concentrated with nitrogen gas and then fractionated with water and hexane. A hexane layer was concentrated to obtain fatty acid ester 1a (crude fatty acid ester).

### Preparation of (S)- and (R)-MTPA Esters (1b and 1c)

Fatty acid ester 1a obtained by methanolysis was divided into two, and each solvent was removed with nitrogen gas. Each vial was added with some 4-dimethylaminopyridine (DMAP) and 80 μL of anhydrous pyridine and then stirred at room temperature for 5 minutes. Then, R-(−) or S-(+)-α-methoxy-α-(trifluoromethyl)phenylacetyl chloride (MTPA-Cl) was added in 5 μL each, and then stirred at room temperature for 16 hours. The reactant was concentrated with nitrogen gas at 40° C., dissolved in methylene chloride (MC), and washed with a 1N HCl solution, a saturated sodium bicarbonate (NaHCO₃) aqueous solution, and brine. The MC layer was treated with anhydrous magnesium sulfate (MgSO₄). The extract was concentrated under reduced pressure to obtain (S)-MTPA ester 1b (0.2 mg, t_{R} 39.4 min) and (R)-MTPA ester 1c (0.3 mg, t_{R} 39.6 min) using reversed-phase HPLC (YMC-Triart C₁₈, 250×4.6 mm, 5 μm, 1.0 mL/min, UV: 210 and 254 nm) and a MeCN—H₂O solvent system with a gradient (40% MeCN 5 min, 40-100% MeCN 30 min, 100% MeCN 10 min). ¹H chemical shift values around the stereogenic center of each MTPA ester were confirmed through ¹H and COSY spectra.

S-MTPA ester of 1a (1b): ¹H NMR (600 MHz, CDCl₃) δ_{H} 5.45 (m, H-3), 3.57 (s, OCH₃), 2.62 (dd, J=15.9, 8.0 Hz, H-2a), 2.56 (dd, J=15.9, 5.0 Hz, H-2b), 1.72 (m, H-4a), 1.64 (m, H-4b); ESIMS m/z [M+Na]⁺497.3.

R-MTPA ester of 1a (1c): ¹H NMR (600 MHz, CDCl₃) δ_{H} 5.45 (m, H-3), 3.64 (s, OCH₃), 2.67 (dd, J=15.9, 8.3 Hz, H-2a), 2.60 (dd, J=15.9, 4.6 Hz, H-2b), 1.63 (m, H-4a), 1.58 (m, H-4b); ESIMS m/z [M+Na]⁺497.2.

### Determination of the Structures of Compounds

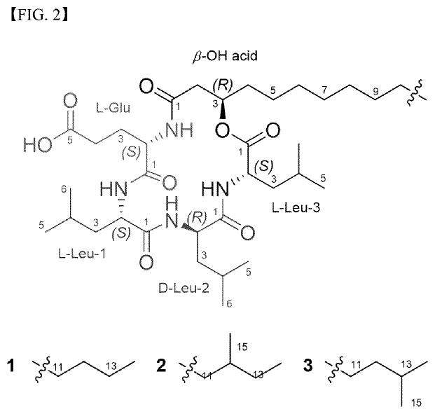

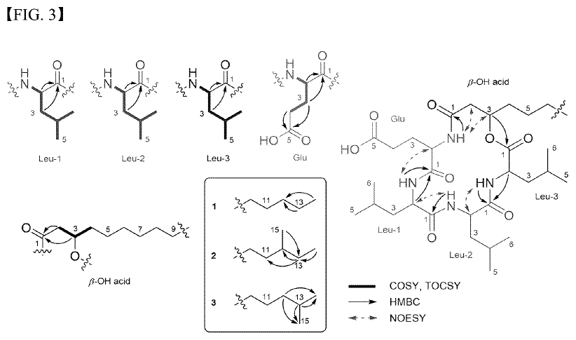

It was confirmed that bacilotetrin C (1) was an amorphous solid, and the molecular formula was C₃₇H₆₆N₄O₈ (unsaturation degree 7) through HR-ESIMS. NMR data of Compound 1 are shown in Table 2 above. In a ¹H NMR (CD₃OH) spectrum, it was confirmed that there were four NH groups (δ_{H} 9.10, 8.39, 7.76 and 7.74). In ¹H and ¹³C NMR and HSQC spectra, four α-protons (δ_{H} 4.57, 4.43, 4.11, and 3.74), long fatty acids (δ_{H} 1.29), oxygen-bonded hydrogen (δ_{H} 5.16), seven methyl hydrogens (δ_{H} 0.96 to 0.89) and six carbonyl carbons (δ_{C} 176.3, 175.9, 174.5, 173.9, 173.3, and 172.9) were confirmed. Through detailed ¹H-¹H COSY, TOCSY, and HMBC spectral analysis, the presence of three leucines (Leu), one glutamic acid (Glu), and β-hydroxy fatty acid (β-OH acid) was confirmed. Based on the degree of unsaturation and molecular formula, the structure of Compound 1 was confirmed as a cyclic lipodepsipeptide (see FIG. 2). The linkage between each amino acid and fatty acid was determined by HMBC and NOESY spectral analysis. The linkage of the peptide was confirmed by HMBC signals (C-1 (δ_{C} 172.9) of Leu-3 in H-3 (δ_{H} 5.16) of β-OH acid; C-1 (δ_{C} 173.9) of Leu-3 in NH (δ_{H} 7.76) and H-2 (δ_{H} 4.57) of Leu-3; C-1 (δ_{C} 173.9) of Leu-1 in NH (δ_{H} 7.74) of Leu-2; C-1 (δ_{C} 175.9) of Glu in NH (δ_{H} 9.10) and H-2 (δ_{H} 3.74) of Leu-1; and C-1 (δ_{C} 173.3) of β-OH acid in NH (δ_{H} 8.39) of Glu). The linkage between amino acid residues and β-OH acid was also supported by NOESY signals (NH (δ_{H} 7.76) of Leu-3 and H-2 (δ_{H} 4.43) of Leu-2; NH (δ_{H} 7.74) of Leu-2 and H-2 (δ_{H} 3.74) of Leu-1; NH (δ_{H} 7.74) of Leu-1 and H-2 (δ_{H} 4.11) of Glu; and NH (δ_{H} 8.39) of Glu and H-3 (δ_{H} 5.16) of β-OH acid). The sequence of compound 1 was determined as cyclo-(β-OH acid-Glu-Leu-1-Leu-2-Leu-3) by the HMBC signal from the α-proton and amide proton of each amino acid to the carbonyl carbon of adjacent amino acids or β-OH acid and the NOESY spectrum between the α-proton and amide proton of amino acids (see FIG. 3). The β-OH acid chain was expected to be linear with one remaining methyl group (δ_{C} 14.5, δ_{H} 0.89), and also consistent with chemical shift values (δ_{C} 14.4, δ_{H} 0.90) of a methyl group of a terminal of linear β-OH acid recently reported and the expectation was supported.

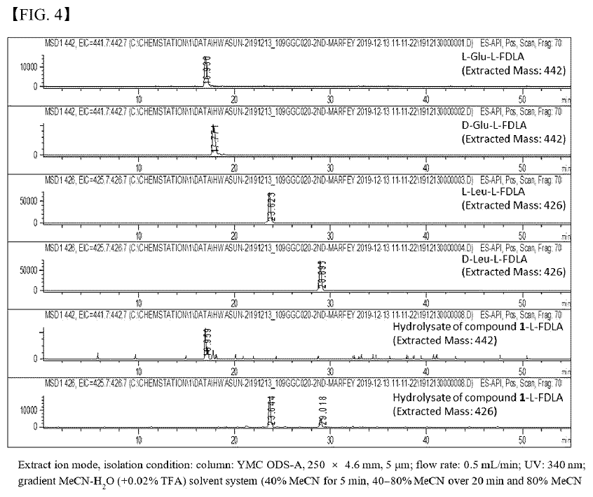

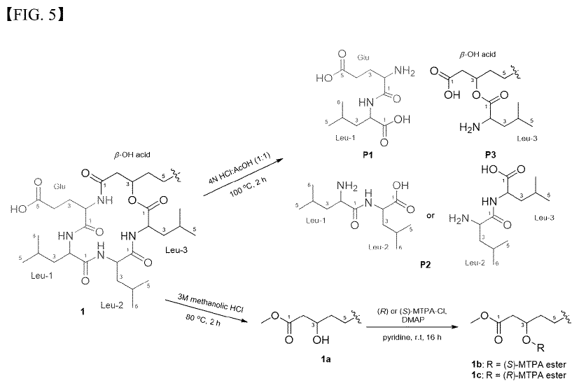

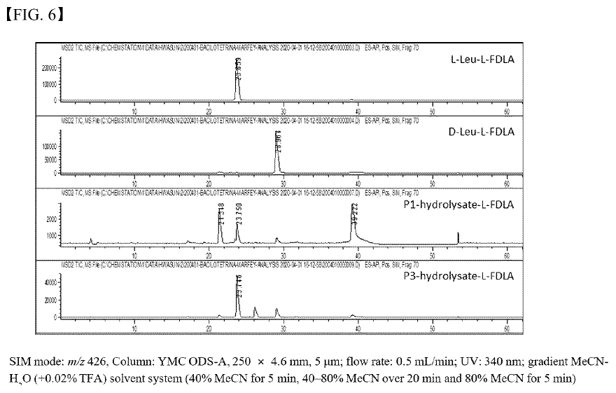

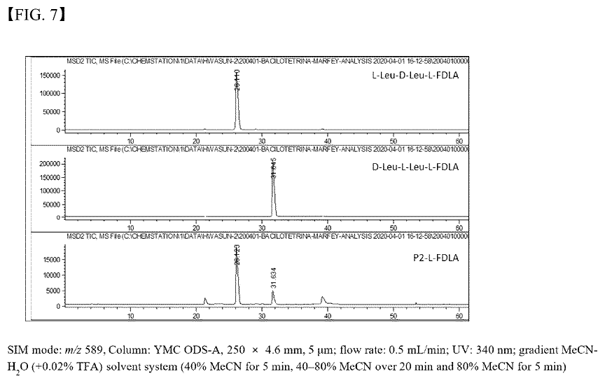

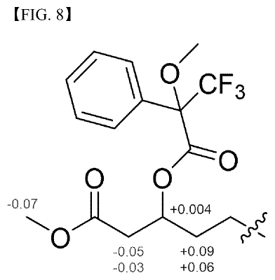

The absolute structure of amino acids included in Compound 1 was determined using an advanced Marfey's method. As a result, it was confirmed that Compound 1 had two L-Leu, one D-Leu, and one L-Glu (see FIG. 4). To confirm the location of D-Leu, Compound 1 was partially hydrolyzed to obtain a dipeptide mixture. HPLC-MS was used to purify the mixture, and as a result, Leu-Glu, Leu-Leu, and β-OH acid-Leu fragments were separated. Both the Leu-Glu and β-OH acid-Leu parts were confirmed as L-form amino acids by Marfey's method (see FIG. 6). Leu-Leu (P2) was obtained by reacting the standard reagents L-Leu-D-Leu and D-Leu-L-Leu with 1-fluoro-2,4-dinitrophenyl-5-L-leucine amide (L-FDLA), respectively, and the retention times were compared and analyzed. As a result of HPLC-MS analysis of the Leu-Leu (P2) portion reacted with L-FDLA, both a large peak (t_{R} 26.1 min, m/z 589 [M+H]⁺) and a small peak (t_{R} 31.6 min, m/z 589 [M+H]⁺) appeared, which were consistent with L-Leu-D-Leu-L-FDLA (t_{R} 26.1 min, m/z 589 [M+H]⁺) and D-Leu-L-Leu-L-FDLA (t_{R} 31.6 min, m/z 589 [M+H]⁺) reacted with standard reagents, respectively (see FIG. 7). Accordingly, the Leu-Leu fragment (P2) was identified as a mixture of Leu-1-Leu-2 (major) and Leu-2-Leu-3 (minor) fragments, and the sequence of three leucines was determined to be L-D-L. To investigate the absolute configuration of β-OH acid, compound 1 was subjected to methanolysis to obtain β-OH acid methyl ester (1a) (see FIG. 5). Compound 1a was divided into two parts, respectively, and attached with (R)-(−)-α-Methoxy-α-(trifluoromethyl)phenylacetyl chloride ((R)-MTPA-Cl) and (S)-(+)-α-Methoxy-α-(trifluoromethyl)phenylacetyl chloride ((S)-MTPA-Cl) to be converted to (S)-MTPA ester (1b) and (R)-MTPA ester (1c). ¹H NMR chemical shift values of Compounds 1b and 1c were confirmed by analysis of COSY spectra. Based on Δδ_{H} values (δ_{S-ester}−δ_{R-ester}), the absolute configuration of C-3 in β-OH acid methyl ester was confirmed as R-form (see FIG. 8).

Bacilotetrin D (2) and E (3) were separated as amorphous solids, respectively, and confirmed to have the same molecular formula as C₃₈H₆₈N₄O₈ (7 degrees of unsaturation) by HR-ESIMS data analysis. NMR data of compounds 2 and 3 were shown in Table 2 above. ¹H and ¹³C NMR spectra of Compounds 2 and 3 were very similar to those of Compound 1, and there was a difference that only a terminal portion of β-OH acid was different. A significant difference in the ¹H and ¹³C NMR spectra of compounds 2 and 3 was in the chemical shift value of the methyl group present at the end of the β-OH acid chain. Bacilotetrin D (2) was a signal of an anteiso-methyl group (δ_{C} 19.7/δ_{H} 0.85 and δ_{C} 11.8/δ_{H} 0.87), whereas bacilotetrin E (3) was a signal of an iso-methyl group (δ_{C} 23.1/δ_{H} 0.87×2). In the HMBC spectrum of compound 2, the linkage to C-11 (δ_{C} 30.7) and C-12 (δ_{C} 35.7) in H-13 (δ_{H} 1.29 and 1.09) of β-OH acid and the linkage to C-13 (δ_{C} 37.8) in H-14 (δ_{H} 0.87) and H-14 (δ_{H} 0.87) were shown. In these signals, it was confirmed that the terminal portion of the fatty acid of Compound 2 was an anteiso-type. Bacilotetrin E (3) was identified as a fatty acid having an iso-methyl terminal by showing a signal from H-14 and H-15 (δ_{H} 0.87) to C-13 (δ_{C} 29.2) of β-OH acid and a signal from H-12 (δ_{H} 1.16) to C-14 (δ_{C} 23.1) and C-15 (δ_{C} 23.1) of β-OH acid in an HMBC spectrum (see FIG. 2). In addition, the methyl signals of compounds 2 (anteiso-) and 3 (iso-) coincided with values reported in the literature (anteiso-type: δ_{C} 19.6/δ_{H} 0.86 and δ_{C} 11.8/δ_{H} 0.88; isotype: δ_{C}23.1/δ_{H} 0.88). Thus, the sequences of compounds 1 to 3 were identified as cyclo-(R-β-OH acid-L-Glu-L-Leu-D-Leu-L-Leu).

The structures of compounds 1 to 3 were similar to surfactins. The surfactins are cyclic lipopeptides and consist of seven amino acids (L-Glu-L-Leu-D-Leu-L-Val-L-Asp-D-Leu-L-Leu) and β-OH acid having 13 to 15 carbon atoms. Compounds 1 to 3 were also cyclic lipopeptides consisting of four amino acids (L-Glu-L-Leu-D-Leu-L-Leu) and β-OH acid having 14 or 15 carbon atoms, in a similar manner thereto. In the structural similarity, it was expected that compounds 1 to 3 were biosynthesized by a biosynthetic pathway (non-ribosomal peptide synthetase, NRPS) similar to surfactins. The cyclic lipopeptides, surfactins, were synthesized by three surfactin synthetase subunits SrfA-A, SrfA-B, and SrfA-C. Among these subunits, SrfA-A and SrfA-B each consisted of three modules, and SrfA-C consisted of one module, and each module was involved in the production of one amino acid. In the case of compounds 1 to 3, it is expected to be a structure generated by inactivating the three modules of the SrfA-B subunit.

### Experimental Example

### Measurement of Mycoplasma Inhibitory Activity

For compounds 1 to 3, the inhibitory activity against Mycoplasma hyorhinis was evaluated using a broth dilution assay. Briefly, an experimental strain Mycoplasma hyorhinis ATCC 17981 was cultured in a PPLO liquid medium at 37° C. in a 5% CO₂ incubator. Compounds 1 to 3 were dissolved in DMSO, serially diluted 2-fold in the PPLO liquid medium to make a concentration of 500-1 μg/mL, and the final DMSO concentration did not exceed 5%. 100 μL of the culture medium, which made with the strain suspension at approximately 2×10⁴ CFU/mL, was added to each well of a 96-well plate. The plates were incubated for 7 days in a 37° C., 5% CO₂ incubator. As the bacteria grew, the medium turned yellow. A minimum inhibitory concentration (MIC) was determined as the lowest concentration at which bacteria did not grow. BioMycoX® (CellSafe, Korea) was used as a positive control. The measurement results were shown in Table 3 below.

Referring to Table 3 below, it was found that the MIC values for Mycoplasma bacteria of compounds 1 to 3 were 31 μg/mL. These results confirmed that the cyclic lipodepsipeptide structure played an important role in the inhibitory activity against M. hyorhinis, rather than the influence of the terminal portion of β-OH acid.

TABLE 3

Compounds

| | 1 | 2 | 3 | BioMycoX ®¹ |
|---|---|---|---|---|
| MIC (μg/mL) | 31 | 31 | 31 | 62 |

¹Positive control.

As described above, the present disclosure has been described through Examples. Those skilled in the art will be able to understand that the present disclosure may be easily executed in other detailed forms without changing the technical spirit or an essential feature thereof. Therefore, it is to be understood that the above-described embodiments are illustrative and not restrictive in all respects. The scope of the present disclosure is represented by claims to be described below rather than the detailed description, and it is to be interpreted that the meaning and scope of the claims and all the changes or modified forms derived from the equivalents thereof come within the scope of the present disclosure.

### [Accession Number]

Depositary Authority Name: Korea Research Institute of Bioscience and Biotechnology

Accession number: KCTC 12411BP

Accession Date: 20130524

What is claimed is:

1. A compound of Chemical Formula 1 below, an optical isomer thereof or a pharmaceutically acceptable salt thereof: 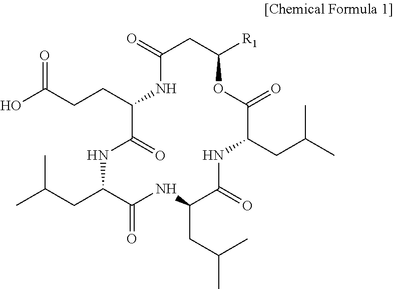
In Chemical Formula 1,
R₁ is hydrogen, straight or branched C₁₋₂₀ alkyl, straight or branched C₁₋₂₀ alkenyl, straight or branched C₁₋₂₀ alkynyl, C₁₋₂₀ alkoxy, C₁₋₂₀ thioalkoxy, C₃₋₂₀ cycloalkyl, C₃₋₂₀ heterocycloalkyl, C₃₋₂₀ heteroaryl, phenyl, or halogen.

2. The compound of claim 1, wherein the compound of Chemical Formula 1 is a compound represented by Chemical Formula 2 below, an optical isomer thereof or a pharmaceutically acceptable salt thereof: 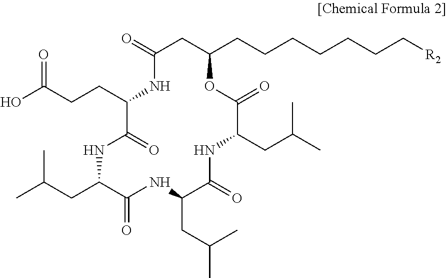
In Chemical Formula 2,
R₂ is hydrogen, straight or branched C₁₋₁₃ alkyl, straight or branched C₁₋₁₃ alkenyl, straight or branched C₁₋₁₃ alkynyl, C₁₋₁₃ alkoxy, C₁₋₁₃ thioalkoxy, C₃₋₁₃ cycloalkyl, C₃₋₁₃ heterocycloalkyl, C₃₋₁₃ heteroaryl, phenyl, or halogen.

3. The compound of claim 1, wherein the compound of Chemical Formula 1 is isolated from a microorganism deposited under Accession No. KCTC 12411BP.

4. The compound of claim 1, wherein the compound of Chemical Formula 1 is selected from the group consisting of compounds 1 to 3 shown in Table below:

| Compound | Structure |
|---|---|
| 1 | 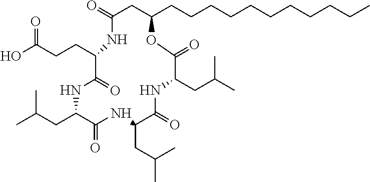 [SMILES: CCCCCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |
| 2 | 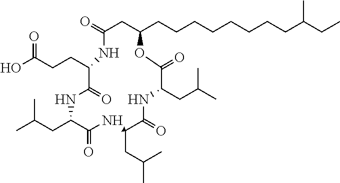 [SMILES: CCC(C)CCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |
| 3 | 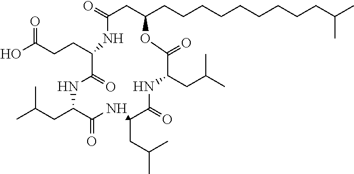 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](CC(C)C)C(=O)O1] |

5. A method for preparing a compound of Chemical Formula 1, an optical isomer thereof, or a pharmaceutically acceptable salt thereof, comprising isolating a compound of Chemical Formula 1 below, an optical isomer thereof, or a pharmaceutically acceptable salt thereof from a Bacillus subtilis 109GGC020 KCTC 12411BP strain or a culture thereof: 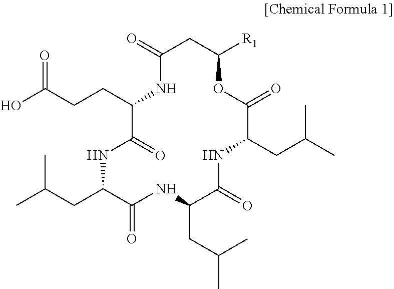
In Chemical Formula 1,
R₁ is hydrogen, straight or branched C₁₋₂₀ alkyl, straight or branched C₁₋₂₀ alkenyl, straight or branched C₁₋₂₀ alkynyl, C₁₋₂₀ alkoxy, C₁₋₂₀ thioalkoxy, C₃₋₂₀ cycloalkyl, C₃₋₂₀ heterocycloalkyl, C₃₋₂₀ heteroaryl, phenyl, or halogen.

6. An antibacterial composition comprising the compound according to any one of claims 1 to 4, an optical isomer thereof, or a pharmaceutically acceptable salt thereof as an active ingredient.

7. The antibacterial composition of claim 6, wherein the compound has antibacterial activity against Mycoplasma.

8. The antibacterial composition of claim 7, wherein the compound has antibacterial activity against Mycoplasma hyorhinis.

9. The antibacterial composition of claim 6, wherein the composition is a pharmaceutical composition.

10. The antibacterial composition of claim 6, wherein the composition is a food composition.

11. The antibacterial composition of claim 6, wherein the composition is a cosmetic composition.

12. The antibacterial composition of claim 6, wherein the composition is a feed composition.

13. A method for preventing or treating infectious diseases caused by microorganisms to be antibacterial of the antibacterial composition, comprising administering the antibacterial composition according to claim 6 in a therapeutically effective dose to a subject other than a human.

14. The method for preventing or treating infectious diseases caused by microorganisms to be antibacterial of the antibacterial composition of claim 13, wherein the infectious diseases caused by the microorganisms to be antibacterial target of the antibacterial composition are at least one selected from the group consisting of polyserositis, arthritis, conjunctivitis, otitis, septicaemia, pneumonia, peritonitis, pleuritis, pericarditis, and porcine reproductive and respiratory syndrome (PRRS).

15. The method for preventing or treating infectious diseases caused by microorganisms to be antibacterial of the antibacterial composition of claim 13, wherein the subject is a pig.

16. A method for sterilization or bacteriostatic of microorganisms to be antibacterial of the antibacterial composition, comprising treating the antibacterial composition according to claim 6 in vitro.
